(19) United States
(12) Patent Application Publication
(10) Pub. No.: US 2016/0061843 A1
(43) Pub. Date: Mar. 3, 2016

(54) BIOMARKER ASSAY

(71) Applicant: Respiratory Clinical Trials Ltd, London (GB)

(72) Inventor: Brian Leaker, London (GB)

(21) Appl. No.: 14/839,019

(22) Filed: Aug. 28, 2015

Related U.S. Application Data
(60) Provisional application No. 62/043,456, filed on Aug. 29, 2014.

(30) Foreign Application Priority Data
Aug. 29, 2014 (GB) 1415343.1

(51) Int. Cl. G01N 33/68

(52) U.S. Cl. CPC G01N 33/6872

(57) ABSTRACT

Methods for measuring the phosphorylation of Signal Transducer and Activator of Transcription (STAT) proteins in sputum, and the application of such methods in evaluating therapeutic agents are provided.

### CROSS REFERENCE TO RELATED APPLICATIONS

This application claims benefit of and priority to U.S. Provisional Patent Application No. 62/043,456 filed on Aug. 29, 2014 and Great Britain Application No. GB 1415343.1, each of which is specifically incorporated by reference in its entirety.

### FIELD OF THE INVENTION

The invention generally relates to biomarker methodology performed on sputum, using, for example, a flow cytometry-based method for measuring the phosphorylation of Signal Transducer and Activator of Transcription (STAT) proteins.

### BACKGROUND OF THE INVENTION

The regulation of protein function in mammalian cells is controlled via reversible protein phosphorylation mediated by protein kinases. Kinases, of which there are over 500 types, are the enzymes responsible for critical signaling pathways in all cell types.

Kinase inhibitors are useful targets for anti-inflammatory diseases, oncology and other areas of medicine, such as autoimmunity and transplantation. Kinase inhibitors are not specific for a single kinase, but have a broad range of activity against multiple kinases. Kinase inhibitors may be selective or non-selective against kinase targets.

Cytokines are the hormonal messengers responsible for cell growth and differentiation, host defense and immunoregulation, including cell-mediated immunity and allergic type responses. One large subgroup, the type I and II cytokine family, encompasses receptors that bind interferons (IFNs), interleukins (ILs) and colony stimulating factors (CSFs). These cytokines all use a common method of signal transduction, namely the Janus kinase-STAT (JAK-STAT) pathway (O'Shea J J et al., N Engl J Med. 2013; 368:161-70).

JAKs are non-receptor tyrosine kinases activated by various cytokine receptors and regulate gene expression through phosphorylation of seven STAT proteins. JAK1/3 heterodimers regulate T cell survival, whereas JAK2 mediates granulocyte-macrophage CSF-mediated neutrophil survival in addition to IFN-gamma (IFNγ) and IL-12/IL-23 signaling. STAT4 is activated by IL-12 and IL-23. STAT3 (and its downstream genes) is activated in lung parenchyma of chronic obstructive pulmonary disease (COPD) patients.

The p38 mitogen-activated protein kinase (MAPK) pathway (see FIG. 1) is activated by a wide range of extracellular stimuli. p38 kinases become activated by phosphorylation via upstream MAPK kinases (MAPKKs; MKKs), which in turn triggers activation of downstream substrates. MAPK-activated protein kinase 2 (MAPKAPK2; MK2) is a p38-activated serine (Ser)/threonine (Thr)-protein kinase involved in cytokine production, inflammatory responses, endocytosis, reorganisation of the cytoskeleton, cell migration, cell cycle control, chromatin remodeling, DNA damage response and transcriptional regulation.

Following stress, MK2 becomes activated via phosphorylation at Thr25, Thr222, Ser272, and Thr334 by p38MAPK, which in turn leads to translocation to the nucleus and direct phosphorylation of a range of substrates. Phosphorylated MK2 is involved in the inflammatory response and acts by regulating tumor necrosis factor alpha (TNFα) and IL-6 production. MK2 also controls the phosphorylation of heat shock protein 27 (HSP27), which can lead to fibrosis. p38α/MK2 is ubiquitously expressed throughout the body with high levels in leukocytes, including inflammatory macrophages.

STAT phosphorylation can be detected easily by Western blotting, but this cannot identify activation in specific cell types in a mixed population. Flow cytometry has been used to detect intracellular STAT1 phosphorylation in whole blood assays and peripheral blood mononuclear cells (PBMC) (Vakkila J et al., Scand J Immunol. 2008 January; 67(1):95-102; Maródi L et al., Clin Exp Immunol. 2001 December; 126(3):456-60), but not in sputum.

The selective JAK inhibitor, tofacitinib, inhibits JAK1, JAK3 and, to a lesser extent, JAK2, but it also inhibits other kinase systems, for example, tyrosine kinase 2 (TYK2). This drug has been approved for clinical use for the treatment of rheumatoid arthritis. It has also demonstrated anti-inflammatory activity associated with clinical improvement in patients with inflammatory bowel disease, psoriasis and renal transplantation in various ongoing clinical studies.

JAK inhibitors, however, are associated with significant adverse effects, especially when used in higher doses. These complications include infections, particularly tuberculosis, hyperlipidemia and a range of bone marrow abnormalities, such as anaemia, that directly result from JAK2 inhibition. These complications limit the amount of drug that can be delivered orally.

In early studies whole blood assays were used to establish the mechanism of action of these drugs to inhibit the STAT phosphorylation pathway in leucocytes (whole blood and PBMCs). It was assumed that these drugs directly inhibit neutrophils, and therefore neutrophil mediated inflammation, via this pathway.

Other more recent compounds in development include pan-JAK inhibitors that have a rapid systemic clearance and so, when inhaled, may maximize local anti-inflammatory activity while minimizing systemic adverse events. Inhaled drugs may be the preferred route of administration for the treatment of inflammatory lung diseases, for example, chronic obstructive pulmonary disease (COPD), IPF, and other inflammatory conditions of the lung.

COPD is an inflammatory disease of the airways characterized by shortness of breath, inflammation and increased levels of pro-inflammatory markers. COPD is also characterized by increased sputum production in certain patients having increased numbers of inflammatory immune cells including neutrophils and macrophages. The numbers of macrophages in the lung are far greater in COPD than, for example, asthma (Barnes P J. Nat Rev Immunol. 2008; 8:183-92). Lung macrophages have a fundamental role in COPD through the release of chemokines that attract polymorphonuclear neutrophils (PMN), monocytes and T cells (Th1 cells; Barnes P J. COPD. 2004; 1; 59-70). In COPD the CD4+ T cells that accumulate in the airway and lungs are Th1 type.

In this latter regard, T lymphocytes are a major source of cytokines. These cells bear antigen specific receptors on their cell surface to allow recognition of foreign pathogens. They can also recognise normal tissue during episodes of autoimmune diseases. There are two main subsets of T lymphocytes, distinguished by the presence of cell surface molecules known as CD4 and CD8. T lymphocytes expressing CD4 are also known as helper T cells, and these are regarded as being the most prolific cytokine producers. This subset can be further subdivided into Th1 and Th2, and the cytokines they produce are known as Th1-type cytokines and Th2-type cytokines. Th1-type cytokines tend to produce the pro-inflammatory responses responsible for killing intracellular parasites and for perpetuating autoimmune responses. IFNγ is the main Th1 cytokine. The Th2-type cytokines include IL-4, IL-5, and IL-13, which are associated with the promotion of IgE and eosinophilic responses in atopy, and also IL-10, which has more of an anti-inflammatory response.

Sputum neutrophils have been correlated with COPD disease progression and established as a primary biomarker of disease activity. Other biomarkers identified in sputum, such as IL-8, Clara cell secretory protein (CC-16) and others, have been associated with disease activity and correlate with disease progression (Dickens J A et al., Respir Res. 2011 November; 12:146; Kim D K et al., Am J Respir Crit Care Med. 2012 December; 186(12):1238-47).

COPD is also associated with an increase in IFNγ production. This increase has been shown to be systemic in some instances, though more characteristically the increase is seen in sputum and bronchial alveolar lavage (BAL) samples. IFNγ decreases phagocytosis and increases inflammatory mediator release from macrophages. IFNγ activates the JAK/STAT signaling pathway via phosphorylation of STAT1. IFNγ may also be the cause of further release or up-regulation of pro-inflammatory cytokines, such as chemokine (C—X—C motif) ligand 9 (CXCL9), CXCL10 and CXCL11 from airway epithelial cells (Barnes P J. J Clin Invest. 2008 November; 118(11):3546-56).

JAKs are a family of enzymes which can catalyze the phosphorylation of various proteins, including STAT1. Gene association studies have found an association between STAT1 and COPD. Upon phosphorylation, STAT1 increases transcription and expression of inflammatory biomarkers (Barnes P J et al. Am J Respir Crit Care Med. 2006 July; 174(1):6-14; Barnes P J. Pharmacol Rev. 2004 December; 56(4):515-48). The JAK/STAT pathway can be activated by IFNγ, and JAK inhibitors are being developed with a view to inhibiting this pathway and thereby reducing airway inflammation Inhibition of this pathway reduces inflammatory mediator release and improves macrophage phagocytosis of bacteria.

IPF is a fatal, chronic, progressive, fibrosing, interstitial pneumonia of unknown cause (ATS/ERS 2002). The lung tissue of IPF patients demonstrates juxtaposition of activated myofibroblast accumulation (fibroblastic foci) and normal lung architecture. IPF clinically presents as a combination of inflammation and fibrosis via immune activation and cyclic acute stimulation of fibroblasts. Targeting myofibroblast accumulation, extracellular matrix production, cell contractility and invasive capacity is expected to reduce fibrosis. Direct targeting of transforming growth factor beta (TGF-β) has not been fruitful due to its central roles in host defense and tumour surveillance. Targeting a distal node in the TGF-β pathway, thus disarming myofibroblast function but avoiding off-target effects, represents an attractive treatment approach. One distal target is MK2.

MK2 inhibitor compounds have potential activity as an inhaled anti-inflammatory therapy for use in chronic inflammatory conditions of the airways. Targeting various steps in the p38MAPK pathway, such as MK2, could lead to a reduction in such biomarkers as TNFα and HSP27 with a possible reduction of inflammation and fibrosis.

### SUMMARY OF THE INVENTION

An assay system to measure STAT phosphorylation in a sputum sample using flow cytometry is provided. The measurement of STAT phosphorylation in sputum by flow cytometry enables direct assessment of the efficacy and sensitivity of therapeutics for the treatment of lung diseases. For example, the efficacy and sensitivity of kinase inhibitor compounds, particularly those delivered via pulmonary administration, for the treatment of lung diseases can be determined using the disclosed system and assay methods. The use of STAT phosphorylation as a biomarker also enables the evaluation of a suitable dosage regimen for a given kinase inhibitor. Furthermore, establishing an intracellular flow cytometry method for sputum allows for identification of specific cell populations expressing phosphorylated STAT (pSTAT), something which has not been previously achievable using the known Western blotting-based methods.

One embodiment provides a method for measuring STAT phosphorylation in a sputum sample using flow cytometry.

Another embodiment provides a method for evaluating the efficacy and/or sensitivity of a kinase inhibitor, the method comprising measuring STAT phosphorylation in a sputum sample using flow cytometry.

Still another embodiment provides a method for evaluating a suitable dose range and/or dosage regimen for a kinase inhibitor, the method including measuring STAT phosphorylation in a sputum sample using flow cytometry.

Another embodiment provides the use of pSTAT as a biomarker for evaluating (i) the efficacy and/or sensitivity of a kinase inhibitor, and/or (ii) a suitable dose range and/or dosage regimen for a kinase inhibitor, the use including measuring STAT phosphorylation in a sputum sample using flow cytometry.

### BRIEF DESCRIPTION OF THE DRAWINGS

FIG. 1 is an illustration of the p38MAPK pathway. This complex pathway includes many branches, and cross talk with other pathways that can regulate a number of different biological consequences. For example, transcription factors such as STAT1 and STAT3 can control cytokine production and p38 regulated/activated kinase (PRAK) is also involved in HSP27 regulation.

FIG. 2 is the forward scatter/side scatter profile of human sputum cells, showing the gating strategy used during flow cytometry. Debris was gated out (shown as the black population streak at the left-hand side of the profile) and the three distinct populations within P1 gated on with specific interest in P4 containing macrophages. The population (P2) to the immediate left of the macrophages (P4) represents neutrophils, and the small population (P3) at the bottom of the profile is unidentified. Sputum leukocytes gated within P1 were thus separated into neutrophils (P2), unidentified cells (P3) and macrophages (P4).

FIG. 3 is a graph of Mean Fluorescence Intensity (MFI) of unstimulated cells, cells stimulated with 10 μl IFNγ (100 ng/ml) (final concentration 10 ng/ml); cells stimulated with 10 μl IFNγ (100 ng/ml) (final concentration 10 ng/ml) and 10 μl inhibitor of the JAK3-selective inhibitor, PF 956980; final concentration 10⁻⁵ M). MFI was measured by flow cytometry.

FIG. 4 is a graph of MFI of cells as treated in FIG. 3, each data point represents a mean value calculated per subject (n=15). Mean fluorescence intensity (MFI) was measured by flow cytometry.

FIG. 5 is a graph of concentration of IL-1b (pg/ml) in sputum supernatants obtained from the same 15 COPD subjects on three to four repeat visits.

FIG. 6 is a graph of concentration of IL-8 (pg/ml) in sputum supernatants obtained from the same 15 COPD subjects on three to four repeat visits.

FIG. 7 is a graph of concentration of macrophage inflammatory protein (MIP)-1b (pg/ml) in sputum supernatants obtained from the same 15 COPD subjects on three to four repeat visits.

FIGS. 8A-D are graphs showing selected cytokine/chemokine concentrations in induced sputum supernatant. In a separate study to the aforementioned flow cytometry study, induced sputum samples were obtained from 10 COPD subjects (clinical diagnosis: GOLD stage 1). Each sputum sample was divided and half of the sample was processed using the disclosed techniques (“modified”), the other half was processed using the standard techniques known in the art (“standard”). Cytokine/chemokine levels following the two different processing procedures were compared. FIG. 8A shows CCL2 levels. FIG. 8B shows CCL5 levels. FIG. 8C shows CXCL9 levels. FIG. 8D shows IL-6 levels.

FIGS. 9A-C are graphs showing cell viability (%) (FIG. 9A), squamous cell contamination (%) (FIG. 9B), and leucocyte differential counts (%) (FIG. 9C) for the same induced sputum samples as illustrated in FIGS. 8A-D. Cell data following the two different processing procedures were compared (“modified/0.05% DTT” refers to the disclosed processing techniques and “standard/0.1% DTT” refers to the established techniques known in the art).

FIG. 10 is a bar graph showing STAT3 phosphorylation in sputum macrophages following stimulation with IFNγ in the absence or presence of increasing concentrations of a MK2 inhibitor. % stimulation was calculated as stimulated MFI/non-stimulated MFI×100.

FIG. 11A is a line graph showing the stimulation of STAT1(Y701) phosphorylation in macrophages and neutrophils from induced sputum by IFNγ, and inhibition of such phosphorylation after pre-incubation with increasing concentrations of a MK2 inhibitor followed by IFNγ stimulation. FIG. 11B is a bar graph showing phosphorylation of STAT1 (Y701) in macrophages in the presence and absence of the MK2 inhibitor expressed as % stimulation. % stimulation was calculated as stimulated MFI/non-stimulated MFI×100.

### DETAILED DESCRIPTION OF THE INVENTION

Systems and methods for the measurement of STAT phosphorylation in a sputum sample using flow cytometry are provided. The sputum sample may be obtained from an individual, as described in further detail below. Sputum should be freshly obtained directly from an individual, ideally via the method described, and preferably processed within certain time limits to maintain the aspects of sputum cell cytology.

A. Sputum Induction

A suitable sputum sample can be obtained from an individual in accordance with standard and well-established procedures. It is advantageous to use the induced method, rather than use spontaneously produced sputum, as the latter results in lower cell viability (Pizzichini M M et al. Am J Respir Crit Care Med. 1996; 154(4 Pt 1):866-9). As an example, but not intended to be limiting in any way, the following procedure may be followed.

The subject inhales 3% (w/v) saline solution mist through the mouthpiece of an ultrasonic nebulizer for five minutes.

Sputum mobilization techniques are then utilized to assist with the production of a sputum sample such as diaphragmatic breathing, huffs, percussion, vibrations and positive expiratory pressure techniques. The subject is asked to attempt to cough sputum into a sputum collection pot.

Spirometry is used as a safety measurement to ensure lung function is maintained throughout the sputum collection procedure. Forced expiratory volume in one second (FEV₁) is the volume of air that can forcibly be blown out in one second, after full inspiration. Assuming the FEV₁ falls by less than 10% after inhalation of 3% (w/v) saline, the participant will be asked to inhale the next saline concentration (4% (w/v)) and repeat the procedure detailed above.

Again if the FEV₁ falls by less than 10% after inhalation of 4% (w/v) saline, the participant will be asked to inhale the next saline concentration (5% (w/v)) and repeat the procedure detailed above.

The sputum collected after 15 minutes of nebulization (e.g., 3×5 minutes) is suitable for processing in the laboratory for flow cytometric analysis.

The sensitivity of the flow cytometric analysis is proportional to the number of macrophages contained in the sputum cells. It is important to have sufficient macrophages in each sample so as to ensure that there is a distinct population to identify using the cell size and granularity flow cytometric method (X/Y gate system) described herein. The technique described herein enables measurement of STAT phosphorylation in a macrophage population, therefore the macrophage population must be of sufficient size to allow analysis. Too small a population would lead to an indistinguishable cell population on the flow cytometry scatter plot.

When measuring phosphorylation of any STAT protein a sufficient number of sputum cells are therefore required per sample to yield suitable macrophage populations.

To enable optimal STAT phosphorylation analysis in a sputum sample, at least 200,000 cells per condition are required. The term ‘condition’ refers to the experimental or control condition that a pool of cells within the sample is subjected to, as part of the analysis being performed. For example, ‘unstained’, ‘unstimulated’ and ‘stimulated’ are three such conditions described further herein. By way of illustration, therefore, in order to measure STAT phosphorylation in cells stimulated with IFNγ compared to unstimulated controls, the sputum sample should ideally contain at least 400,000 sputum cells (i.e., 200,000 cells for each condition). If two stimulators of STAT phosphorylation were to be assessed alongside a control, the sample would ideally contain at least 600,000 cells, and so on. The sample, once obtained, can therefore be split into the requisite number of pools for the one or more conditions being assessed, each pool containing a sufficient number of cells for STAT phosphorylation analysis to be performed.

In one embodiment, a minimum macrophage count of around 4% allows for accurate gating of the macrophage population. When the macrophage population of the sample is above 4% then a cell count of around 200,000 cells per condition gives a distinct macrophage population allowing for accurate gating. Useful data can be obtained from more and/or less cells per condition, but the best results are obtained when the sample contains around 200,000 cells per condition with at least around 4% macrophages. Sputum samples obtained from certain groups of individuals may contain 100% neutrophils and it has been found that these are not suitable for analysis by the method. This finding also indicates that neutrophils are not the primary cell type of interest in this STAT inflammatory pathway.

Thus, the sputum sample may contain at least 100,000, at least 150,000, at least 200,000, at least 250,000, at least 300,000, at least 350,000 or at least 400,000 sputum cells per condition. There is no upper limit per se, but in some embodiments, the sputum sample contains no more than 500,000, no more than 400,000, no more than 300,000 or no more than 250,000 cells per condition. The sputum sample may contain around 100,000-500,000, around 125,000-325,000 or around 150,000-250,000 cells per condition; preferably it contains around 200,000 cells per condition.

The macrophage population of the sample may be above 1%, above 2% or above 3%, but preferably it is above 4%, and may even be above 5%, above 6%, above 7%, above 8%, above 9%, above 10%, above 15% or above 20%. In a preferred embodiment, the macrophage population is in the region of 3%-20%, or 3%-10%, or 3-6%, most preferably in the region of 4-5% of the sample.

One or more samples may be collected from a subject on repeat visits, for example, two, three, four or more samples may be taken over a period of a number of weeks or months, repeat visits being ideally separated by a minimum of seven days. As many repeat visits as required by the protocol should be allowed. The taking of multiple sputum samples from a subject enables data to be averaged per subject and/or statistically analyzed with confidence, which will improve the quality of the statistical analysis. Serial multiple samples obtained over time also enable STAT phosphorylation levels to be monitored over a defined period.

B. Sputum Processing

The sputum sample can be processed in order to obtain viable cells for analysis free from mucus contamination. Sputum processing can be important for a flow cytometry signal being measured in such samples.

Sputum is a notoriously difficult bodily fluid with which to work. In this regard, the mucus content of sputum contains and shields within it the cells and biomarkers of interest. When sputum is taken out of the body, the cells inside immediately start to die. Any processing of the sputum therefore needs to be harsh enough to break through the mucus shell, yet gentle enough to keep the cells alive.

The processing steps used in the art for measuring STAT phosphorylation are not suitable for sputum. These techniques are performed on whole blood, which contains a different array of cells in a different cellular environment compared to sputum.

Equally, where techniques in the art have been performed on sputum, for example, to measure sputum cell count and/or inflammatory cytokine levels in the sputum fluid phase in health and disease, they have not been designed, and therefore are not appropriate, to measure STAT phosphorylation.

The following procedure as described in the Examples has been shown to be suitable for use in the disclosed methods. The described technique has been modified and adapted from that used by Pizzichini et al. (see, e.g., Pizzichini E et al., Eur Respir J. 1996 June; 9(6):1174-80, Pizzichini M M, et al., Am J Respir Crit Care Med. 1996; 154(4 Pt 1):866-9; designed for sputum, but not to measure STAT phosphorylation), in order to ensure compatibility with the flow cytometric methodology described herein.

Induced sputum is suitably kept on ice and processed as soon as possible after collection, preferably within four hours, even more preferably within three hours, and most preferably within two hours, if not one hour, of collection. Immediate processing is desirable to ensure high cell viability. Sputum plugs are selected for processing and suitably transferred into a centrifuge tube.

The volume of the selected sputum sample is noted and an equal volume of Dulbecco's phosphate buffered saline (DPBS) typically added.

To liquefy the sample, a reducing agent is added. The reducing agent breaks down the thick mucus, allowing the cells inside to become separable therefrom. Any reducing agent may be used, but dithiothreitol (DTT) is preferred. DTT may be provided in any form, including Sputolysin®. The final concentration of reducing agent should be in the range of less than 0.1% (w/v), preferably less than 0.08% (w/v) and more preferably less than 0.06% (w/v). A final concentration of around 0.05% (w/v) is preferred; this concentration of reducing agent was empirically determined to result not only in cells suitable for flow cytometric analysis but also higher yields of biomarkers of interest compared to higher concentrations. This is a significantly lower concentration than is standard in the art for sputum samples.

The tube is then suitably placed on a plate shaker, at a gentle speed in the range of around 150 to around 450 rpm, but preferably around 300 rpm. The tube is shaken at room temperature for a sufficient length of time to disperse the cells without activating any inflammatory cells. For example, anywhere between around 15 minutes and around one hour would be suitable to allow for mucus breakdown, but around 30 minutes is preferred. This incubation time is around 3× longer than standard sputum processing techniques.

The sample is then suitably mixed gently with a Pasteur pipette and left to shake for a further short period of time, such as around 5 minutes to around 30 minutes, and preferably around 15 minutes.

The described sputum processing technique is a much gentler technique than that employed in known sputum assays and sputum processing techniques. Standard sputum processing techniques typically use 0.1% (w/v) DTT, an incubation time of 15 min with centrifugation of 400 G for 10 min at 4° C. The disclosed processing conditions advantageously involve a lower concentration of reducing agent, longer incubation times and gentler sample handling, and are such that cell viability post-sputum processing is at least 70%, preferably at least 80%, and most preferably at least 85% for a typical sample.

The processing technique may also involve protease inhibition of the sputum sample. For example, protease inhibitor can be added to the sample at the time of incubation with the reducing agent, with a view to reducing the damaging effects of proteases present in the sputum sample or released from inflammatory cells activated during the processing method. Any protease inhibitor may be used, but preferably a cocktail protease inhibitor is used, which may include, but is not limited to, 4-(2-Aminoethyl) benzenesulfonyl fluoride hydrochloride (AEBSF), Bestatin, E-64, Pepstatin A, Phosphoramidon, Leupeptin and Aprotinin. Protease inhibitors are commercially available and should be used at the manufacturer recommended concentration. In some embodiments, therefore, the sputum processing step further includes inhibiting any proteases in the sample. It is believed that this additional step may have a beneficial effect on the stimulation/non-stimulation signal separation observed using flow cytometric analysis.

The processed sample may then be separated into its cell and liquid fractions by centrifugation. Centrifugation should be a gentle process, in order to maintain cell viability. For example, the sample can suitably be centrifuged at about 1200 rpm (258 g) for about 10 minutes at room temperature, however, any centrifugation conditions that result in sufficient separation can alternatively be employed.

The cell fraction may then be washed, for example, using DPBS.

Sputum supernatant can be collected and optionally used to measure any biomarkers of inflammation of interest, such as cytokines/chemokines and others including those discussed in more detail below.

By way of example, in some embodiments, the sputum sample is treated with an effective amount of DTT at a concentration of less than 0.1% (w/v) and optionally agitating or shaking the sample under conditions that release the cells from mucus suitable for antibody staining while maintaining viability of at least 50%, 60%, 70%, 80%, 90%, 95% or more of the cells.

C. Cell Counting

The cell pellet is then suitably resuspended in a known volume of DPBS. The cell suspension can be stained with a cell staining agent. For example, staining can be achieved by dilution in 0.4% Trypan blue solution or such like. The sample can then be loaded onto a haemocytometer in order to count the cells using microscopy, in accordance with standard procedures.

For the example provided above, total leucocyte count per millilitre of suspension can be calculated by multiplying the total average leucocyte count by the dilution factor and multiplying by 10⁴.

D. Inducing STAT Phosphorylation

After the sputum has undergone the above-described liquefaction and a total cell count has been performed, the sputum cells are suitably centrifuged. Any conditions resulting in sufficient separation can be employed; exemplary conditions are about 1200 rpm for about 10 minutes at room temperature. The cell pellet is suitably resuspended in DPBS, at a concentration of around 1.5×10⁶ cells/ml to around 2.5×10⁶ cells/ml, but preferably at a concentration of around 2×10⁶ cells/ml. The sample is typically left to rest undisturbed at around 37° C. for approximately one hour.

The cells may then be incubated with a stimulator of STAT phosphorylation. Any such stimulator may be used. In an embodiment, therefore, the method includes inducing STAT phosphorylation with one or more cytokines. Suitable cytokines include, but are not limited to, IFNγ, IFNα, IL-2, IL-3, IL-4, IL-5, IL-6, IL-7, IL-9, IL-10, IL-12, IL-15, IL-23, epidermal growth factor (EGF), platelet derived growth factor (PDGF), GM-CSF, growth hormone, prolactin and erythropoietin. Preferably, IFNγ and/or IL-6 are used. These cytokines are both stimulators of STAT1 in macrophages, but cause different and distinct cellular responses.

The phosphorylation of any STAT protein can be measured using the disclosed methods. In an embodiment, therefore, the method is for measuring STAT1, STAT2, STAT3, STAT4, STAT5A, STAT5B and/or STAT6 phosphorylation. In preferred embodiments, the method is utilized for measuring STAT1 or STAT3 phosphorylation.

Cell stimulation via different cytokines may produce optimal phosphorylation of the different components of the STAT pathway. Different cytokines can therefore be used to detect the different STAT proteins; for example, IFNγ can be used to detect STAT1, as evidenced by Examples 1 and 4, or STAT3, as evidenced by Example 3. Any and all working combinations of STAT phosphorylation stimulators and STAT proteins to be measured are encompassed by the disclosed methods. Phosphorylation may be measured at any amino acid residue in the STAT protein where phosphorylation occurs. Taking STAT1 as an example, phosphorylation therefore may be measured in tyrosine at residue 701 (Y701), serine at residue 272 or threonine at residue 25, 222 or 334, for example. Any phosphorylated residue may thus be targeted when measuring the phosphorylation of a STAT protein.

In some embodiments, the method is for measuring STAT1 phosphorylation induced by IFNγ and/or IL-6. Binding of cytokine to its receptor triggers activation of JAK and subsequent phosphorylation of the cytoplasmic terminal tyrosine residues. The phosphotyrosine interacts with Src Homology 2 (SH2) domains on STATs causing activation, dimerisation, nuclear translocation and transcriptional activation (Ivashkiv L B et al., Arthritis Res Ther. 2004; 6(4):159-68). Fluorescently-labeled antibodies specific for the phosphorylated tyrosine residues on the STAT proteins are commercially available and allow the detection of intracellular pSTAT proteins following stimulation. Each STAT protein can be detected by a single specific antibody, in accordance with manufacturers' instructions (see various manufacturers' websites, e.g. www.bdbiosciences.com).

The cells may therefore be separated into separate pools for alternative treatments (‘conditions’). For example, to assess STAT1 phosphorylation, one pool of cells may be incubated with IFNγ alone, a second pool with IL-6 alone and a third pool with IFNγ and IL-6. Other combinations of cytokines, such as those mentioned above, may be required to stimulate different STAT proteins.

In order to induce STAT phosphorylation, a suitable volume and number of cells should be aliquoted for analysis, into polystyrene flow cytometry tubes or such like. A sample volume in the range of around 50 μl to around 500 μl would be suitable, around 100 μl is preferred. A range in cell number of around 100,000 to around 500,000 would be suitable, around 200,000 cells are preferred.

A suitable amount of a stimulator of STAT phosphorylation is added to each sample. The final concentration is typically in the range of around 1 ng/ml to around 100 ng/ml; around 10 ng/ml is preferred. Thus, as an example, 10 μl IFNγ (100 ng/ml) can be added to each sample (final concentration 10 ng/ml). As a negative control, the same volume of DPBS (for example, 10 μl DPBS) can be added to non-stimulated cells.

In some embodiments, the method includes inducing STAT phosphorylation in the presence of a kinase inhibitor. The kinase inhibitor may be indicated for inhalation, oral or intravenous administration. Any kinase inhibitor may be used, including selective and non-selective protein kinase inhibitors. Such inhibitors include, but are not limited to, Protein Tyrosine Kinase (PTK) inhibitors, which include Src, Csk, Ack, Fak, Tec, Fes, Syk, Abl and Jak inhibitors, the latter including PF 956980 (Axon Medchem), a known JAK3-selective inhibitor. MK2 inhibitors are also included. Inhibition may therefore occur in any STAT phosphorylation pathway; for example, a JAK inhibitor may be used to inhibit phosphorylation via the JAK-STAT pathway and/or a MK2 inhibitor may be used to inhibit phosphorylation via the MAPK pathway. The kinase inhibitor may be indicated for the treatment or prevention of lung disease, preferably inflammatory lung disease, and more preferably lung disease characterized by TH1 inflammatory mechanisms including, but not limited to, COPD, IPF and similar conditions. Suitable methods for inducing STAT phosphorylation in the presence of a kinase inhibitor are described further below.

The samples are then suitably incubated in a water bath at approximately 37° C. for around 20 minutes. Any suitable incubation conditions can alternatively be used.

E. Sample Fixation and Permeabilization

The samples are removed from the water bath and separated into their cell and liquid fractions by centrifugation. The sample can suitably be centrifuged at 258 g for five minutes at room temperature, but any centrifugation conditions that result in sufficient separation can alternatively be employed.

The supernatant is removed and the cell pellet resuspended in a suitable medium. For example, the cell pellet can be resuspended in 100 μl of 4% (w/v) paraformaldehyde in DPBS. The samples can then be incubated in the water bath at approximately 37° C. for around 15 minutes, to fix the cells.

Fixation is an important step as it prevents any further alteration to the cell. Cellular changes brought about during the stimulation step will be permanently ‘fixed’ by the addition of paraformaldehyde and no further changes will occur. Any measurable differences in the state of the cell will therefore be attributable to the stimulation step rather than any subsequent manipulation. The disclosed methods therefore advantageously involve a cell fixation step.

Intracellular flow cytometric analysis also involves a cell permeabilization step. This allows antibodies directed against pSTAT to enter the cell. Upon entering the cell these antibodies, conjugated with a suitable detection system (see section F), bind to the intracellular target pSTAT proteins. The disclosed methods can therefore include a permeabilization step if anti-pSTAT antibodies are to bind to their intracellular target. It has been determined that standard methodologies for permeabilizing cells do not work using this antibody system. Rather, a cell permeabilization technique provided that can be included in the other methods disclosed herein. 100% (v/v) methanol is used for the permeabilization step, which has been determined to result in successful intracellular staining using antibodies directed against pSTAT proteins.

The (flow cytometry) tubes are thus removed from the aforementioned water bath and typically centrifuged at about 258 g for about five minutes at room temperature. Any centrifugation conditions that result in sufficient separation can alternatively be employed. The supernatant is removed and the cell pellet resuspended in, for example, 0.8 ml staining buffer (DPBS+2% human serum).

The tubes are again typically centrifuged at about 258 g for about five minutes at room temperature. The supernatant is removed and the cell pellet resuspended in, for example, 0.35 ml of 100% (v/v) ice-cold methanol (stored at −20° C.). The samples are then suitably incubated on ice for 20 minutes, to permeabilize the cells.

F. Sputum Staining For Flow Cytometry

The tubes are typically centrifuged at about 258 g for about five minutes at room temperature. The supernatant is removed and the cell pellet resuspended in, for example, 0.8 ml staining buffer.

The tubes are again typically centrifuged at about 258 g for about five minutes at room temperature. The supernatant is removed and the tubes blotted dry with laboratory tissue to ensure the removal of most of the liquid.

The cell pellets are resuspended in staining buffer with the addition of a further amount of staining buffer alone, an anti-pSTAT antibody or an isotype control. Any suitable staining buffer may be used, typically a saline solution with up to 10% protein added, preferably DPBS+2% human serum. Any suitable antibody may also be used. Antibodies are commercially available for all seven STAT molecules currently described (Ivashkiv L B et al., Arthritis Res Ther. 2004; 6(4):159-68) conjugated with a variety of fluorescent markers (fluorescein isothiocyanate (FITC), phycoerythrin (PE), peridinin chlorophyll (PerCP), Alexa Fluor® 488 and 647). Suitable volumes will be known to the skilled person. For example, the cell pellets may be resuspended in 100 μl staining buffer with the addition of either 20 μl staining buffer alone (unstained cells) or 20 μl (1.5 μg/ml) Alexa Fluor® 647 conjugated anti-pSTAT1 antibody (PhosFlow, BD Biosciences) (STAT stained cells) or isotype control (control cells) at the same concentration as pSTAT1. The samples are typically incubated at room temperature, covered in foil, for 30 minutes.

A volume of around 0.5 ml to around 4 ml, preferably around 2 ml, staining buffer, can then be added and the tubes suitably centrifuged at 258 g for five minutes at room temperature. Any centrifugation conditions that result in sufficient separation can alternatively be employed. The supernatant can be removed and the cell pellet resuspended in, for example, 500 μl staining buffer, ready for flow cytometric analysis.

Although most of the disclosure herein refers to treatment of the sputum sample or cells thereof with an antibody, it will be appreciated that other suitable detection reagents suitable for flow cytometry are known in the art and can be used in addition or alternative to an antibody in any of the methods disclosed herein.

In addition to antibodies and antigen binding fragments thereof, reagents and ligands used for cell detection by flow cytometry include, for example, but are not limited to, other ligands that bind, preferably bind specifically, to the molecule of interest. For example, the ligand can be a protein, nucleic acid, or small molecule. The ligand is typically labeled with a fluorophore for detection by the flow cytometer. The labeling can be covalent (e.g., a fluorescently labeled primary antibody) or non-covalent (e.g., a fluorescently labeled secondary antibody that binds to a primary detection ligand). In some embodiments, in addition or alternative to a fluorophore, the ligand or reagent can be labeled with a radioisotope, quantum dot, or other suitable molecule.

In preferred embodiments, the ligand is an antibody or antigen binding fragment thereof that binds specifically to pSTAT. As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

By way of non-limiting example, a method for measuring STAT phosphorylation in a sputum sample by flow cytometry can include contacting the sputum sample or cells thereof with an antibody or antigen binding fragment thereof, or another ligand that binds specifically to a phosphorylated STAT, and detecting or measuring the level of antibody or ligand binding by flow cytometry.

G. Flow Cytometry

Equipment and machinery for flow cytometry offered by any manufacturer can be used in the disclosed methods, and operated in accordance with the manufacturer's instructions. In an embodiment fluorescence-activated cell sorting (FACS®) is used. For example, a FACSCanto® II flow cytometer (BD BioSciences, Oxford, UK) may be used.

The use of flow cytometry is advantageous, as there has previously been a paucity of flow cytometric methods used in sputum analysis. It is believe that this paucity may be explained by the fact that DTT cleaves cell surface markers, which renders antibody-based detections systems, which bind to these markers, much less sensitive. The provision of a flow cytometry-based method for measuring STAT phosphorylation in sputum, which can be used sensitively with an antibody-based detection system, is therefore of great value to the industry. It is believed that the flow cytometry-based methods described herein are more sensitive and will result in higher levels of biomarker measurements in sputum samples compared to known methods. The methods advantageously detect intracellular levels of STAT signaling, made possible by the fixation and permeabilisation of cells allowing intracellular binding of antibodies.

A procedure for flow cytometry analysis is described below. The skilled person would readily appreciate how to adapt the following procedure for use with any flow cytometry equipment. Flow cytometry is a laser-based technology that can be used for cell counting, cell sorting, and/or biomarker detection. Flow cytometry generally includes passing a steam of suspended cells past an electronic detection apparatus (e.g., a flow cytometer). Prior to detection, cells are typically contacted with a reagent that labels the cells or a subset thereof. Typically, the disclosed methods include contacting a sputum sample, or cells thereof, with a reagent or ligand that binds to a pSTAT. Preferably the ligand or reagent binds specifically to the pSTAT. Detection of the ligand or reagent during flow cytometry allows the user to detect cells that have pSTAT within or on the surface of cells, and can be used to distinguish them from cells that do not have pSTAT within or on the surface of cells.

Results can merely indicate whether a certain threshold level of detection set by the user is present or absent. In some embodiments, flow cytometry may be used to measure the level of pSTAT expressed by individual cells of the sample. The level can be quantitative or qualitative. The level can be, for example, the mean florescent intensity of the labeled ligand.

The practitioner can use standard analysis techniques to draw conclusions about the level of pSTAT expression in the cells of the sample. In some embodiments, the cells are also contacted with second, third, or more detectable ligands. The second, third or more ligands, can, for example, be used to distinguish between different cell types (e.g., macrophage and neutrophils), live and dead cells (e.g., propidium iodide), or to detect other biomarkers (e.g., cytokines, cell surface receptors, etc.). In some embodiments, STAT and pSTAT are separately detected, allowing the user to determine both the overall level of a STAT relative to its level of phosphorylation in a population or subpopulation of cells.

The data generated by flow cytometers can be analyzed using known techniques. For example, results can be plotted in a single dimension, to produce a histogram, or in two-dimensional dot plots or even in three dimensions. The regions on the plots can be sequentially separated, based on fluorescence intensity, by the user, a preset algorithm, etc., to create a series of subset extractions referred to as “gates.” Such analysis allows the user to characterize the original cell sample into subpopulations based on the detected ligand(s) used. Software for analysis of flow cytometry data is well known in the art, and include, for example, WinMDI, Flowing Software, Cytobank, FCS Express, Flowjo, FACSDiva, CytoPaint (aka Paint-A-Gate), VenturiOne, CellQuest Pro, Infinicyt and Cytospec.

In some embodiments, the cells are sorted into one or more subpopulations by the flow cytometer (e.g., Fluorescence-activated cell sorting (FACS)). Subpopulations can be retained for further analysis by the user.

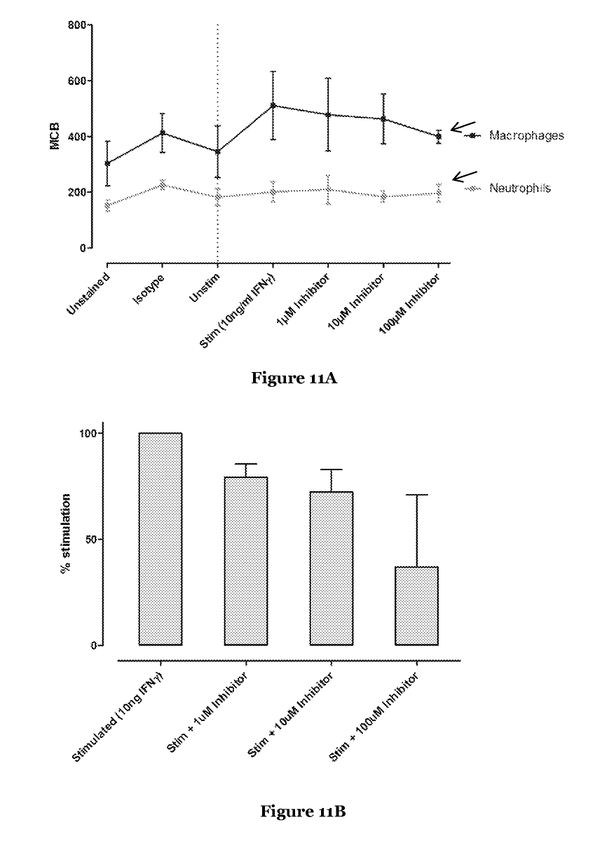

It has been determined, that for STAT1 and STAT3 analysis, the disclosed method works in macrophage populations in sputum, as these cells can produce a STAT1 and STAT3 phosphorylation signal. The method can be applied to all STAT proteins in macrophages, though here it is illustrated by STAT1 and STAT3. Different STAT proteins may be relevant in different disease states and the phosphorylation pathway may be inhibited by different kinase inhibitors (see below). Based upon previously existing knowledge in the art, neutrophils had been the presumed cell of interest. In fact, neutrophils were previously believed to be the important cell type in kinase pathways with particular relevance to inflammation. When pure neutrophil populations were studied, no activation signal could be obtained and pSTAT could not be detected (see, for example, FIG. 11 (Example 4)). The development of the disclosed methods has thus identified that macrophages may actually be the important cell of interest in the STAT1 phosphorylation pathway.

Indeed, where flow cytometry has been used by previously to detect pSTAT1 and/or to assess the efficacy of JAK inhibitors, this was not in relation to lung disease and no measurements were performed in sputum. Rather, they tended to concern hematopoietic and myeloproliferative disorders and, consequently, were heavily focused on taking measurements from samples of blood. They also made no mention of macrophages being the important cell type to study.

Macrophages found in the lung may be resident and proliferate in the lung in response to certain stimuli. It should not always be assumed that PBMCs (monocytes) migrate into the lung from the systemic circulation (Murray P et al., Nat Rev Immunol. 2011; 11(11): 723-737). Resident lung macrophages have been classified as M1 and M2 macrophages (Mantovani A et al., Immunity. 2005; 23(4):344-6) where, broadly speaking, M1 macrophages are pro-inflammatory and M2 macrophages are anti-inflammatory (Mantovani A et al., Immunity. 2005; 23(4):344-6, Kunz L I et al., Respir Res. 2011; 12:34). M1 macrophages are stimulated by IFNγ triggering the release of chemokines CXCL9, CXCL10 and CXCL11 (Mantovani A et al., Immunity. 2005; 23(4):344-6). The effects of developing COPD cause an increase in the number of M2 macrophages. M2 macrophages are highly phagocytic and it has been widely reported that phagocytosis decreases COPD. One explanation of this seeming contradiction could be that the reduced phagocytosis is due to the increased levels of proteases in the lung environment in COPD. This M2 polarization of macrophages results in remodeling of the lung parenchyma. Taken together the polarization of macrophage phenotypes from a steady state to a reduced M1-increased M2 state could be an indication that macrophages are responsible for the remodeling evident in COPD but are less important in the chronic inflammation (Shaykhiev R et al., J Immunol. 2009; 183(4):2867-83).

It has also been deduced that the flow cytometric assay system will detect a STAT1 and STAT3 phosphorylation signal when there is a sufficient number of macrophages present in the sample. The assay can be used to assess all STAT proteins in macrophages, though here it is illustrated by STAT1 and STAT3. A population in the region of 4-5% macrophages in a sputum sample will give a sufficient, distinct macrophage cell population (discussed in more detail above). As a guide, at least 4% macrophages, preferably at least 5% macrophages, more preferably at least 10% macrophages, even more preferably at least 15% macrophages, and most preferably at least 20% macrophages, in a total cell count of 10,000 can be included per flow cytometry sample for STAT analysis. Generally, this ratio is also seen in the sputum cell counts and differential. Flow cytometry may then be used to identify the macrophage population, as described herein. A FACSCanto II® flow cytometer (BD Biosciences, Oxford, UK) is suitable for use in this analysis step.

The volume, cell count and viability of the sputum sample can all contribute to the success of the methods described herein. Ideally the volume of the sputum sample for analysis by flow cytometry should be at least 100 μl, preferably at least 200 μl, more preferably at least 300 μl and most preferably at least 400 μl or even at least 500 μl. Samples of sufficient size and quality can be reliably obtained from COPD and IPF patients, smokers and other such patient groups and populations.

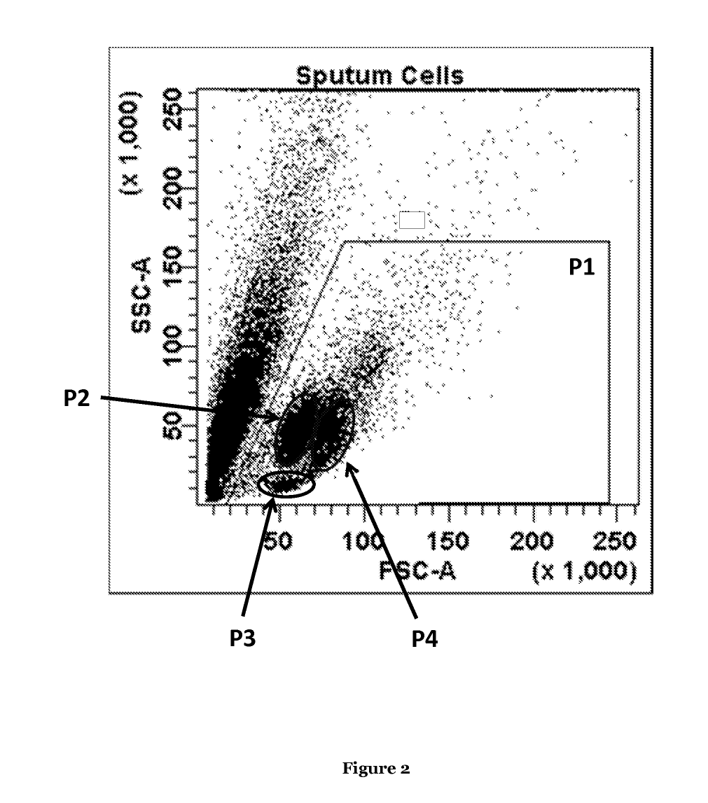

As above, it has been deduced that, for flow cytometric analysis, approximately 200,000 sputum cells per condition with at least 4% macrophages can be included per original sample to yield suitable macrophage populations in the final processed sample for analysis (discussed in more detail above). In this regard, it has been deduced that the assay will not work when a sputum cell population is 100% composed of neutrophils. The data presented in Example 1 confirms this finding using the gating method during flow cytometry. As illustrated in FIG. 2, debris was gated out and the three distinct populations within P1 gated on with specific interest in P4 containing macrophages. Isolating and identifying neutrophils by this method did not show any change in the signal produced by STAT1 phosphorylation. Where sputum samples made of 100% neutrophils were studied no signal change was detected. Indeed, sputum neutrophil count has previously been described as the major biomarker in COPD. The data provided herein conversely indicates that macrophages may emerge the most relevant and important effector cell in lung inflammation in COPD. Hence this observation is believed to have direct implications for drug targets and biomarker interpretation of sputum biomarkers in COPD and other inflammatory conditions of the lung.

IPF represents a more heterogeneous condition where although cough is a frequent clinical symptom it is often non-productive of sputum. In patients who produce sputum, the morphology is similar to that seen in COPD patients (Beeh K M et al., Respir Med. 2003; 97(6):634-9).

Human sputum cell populations can thus be determined by their forward scatter/side scatter profiles. This distinction of separate cell populations via flow cytometric analysis based upon the physical properties of the cells alone is enabled via the use of a lower concentration of reducing agent (such as DTT) compared to known techniques. This also means, therefore, that there is no requirement in the disclosed methods for fluorescent cell surface marker antibodies to pick out the cells of interest.

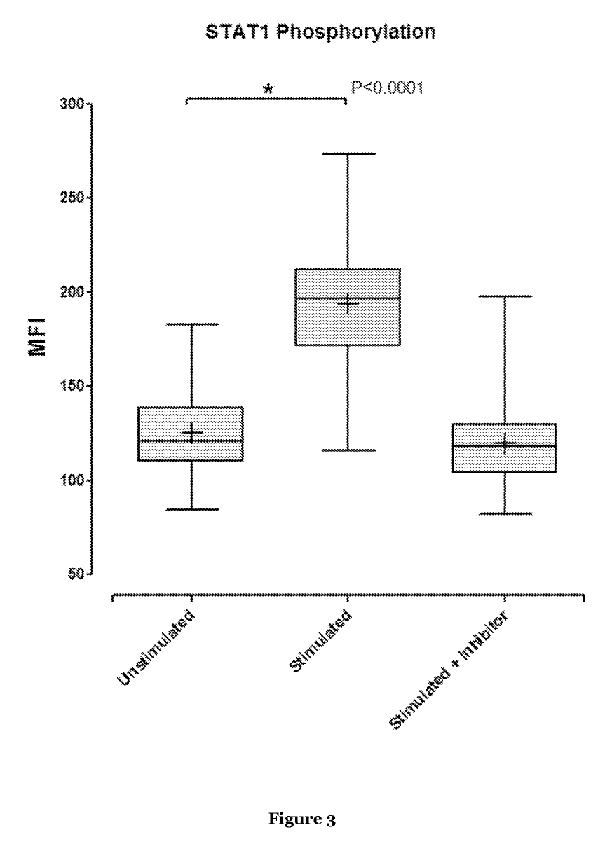

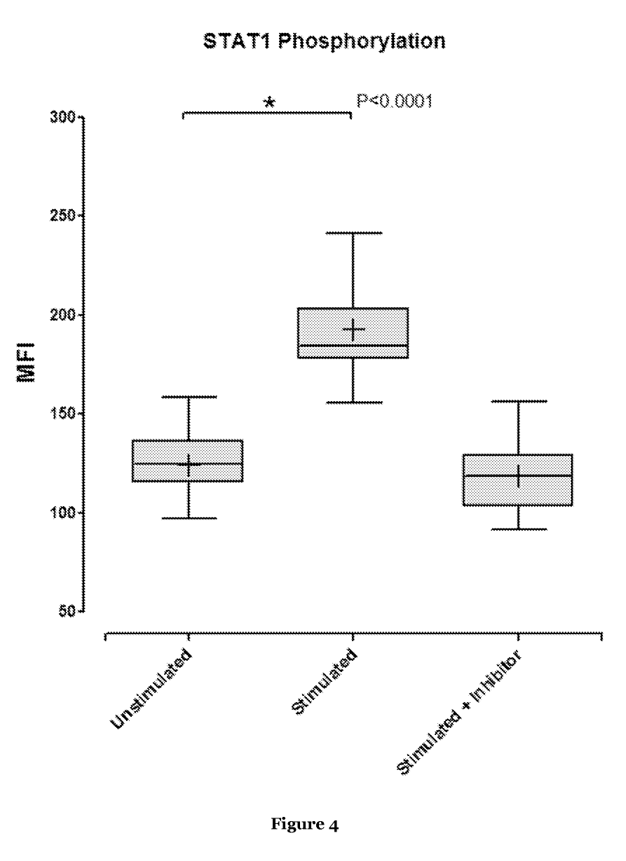

STAT phosphorylation can be measured in a macrophage population by dividing the MFI of the stimulated sample by that for the non-stimulated sample. A value greater than one (>1) indicates positive staining. The working Examples below also show that the methods described herein are able to differentiate between stimulated and unstimulated cells (FIGS. 3 and 4).

Thus, in one embodiment STAT phosphorylation being detected, or not detected, in the sputum sample. The final step of the method may thus be determining the presence or absence of pSTAT in the sputum sample. The final step of the method may be determining the amount of pSTAT in the sputum sample, typically relative to other sputum samples, which, as above, can be expressed in terms of MFI. The MFI is a measure of fluorescence intensity and as such is dependent upon the type of conjugated antibody employed. Although the MFI does not provide a stoichiometric measurement of the number of pSTAT molecules it does enable a direct comparison of two samples stained with the same antibody to be made, with a relative increase in MFI equaling a relative increase in STAT phosphorylation.

H. Analysis of Biomarkers of Inflammation

In an embodiment, the level(s) of one or more biomarkers of inflammation, such as cytokines or chemokines, are quantified in the same sputum sample obtained from the individual. This feature is enabled via the use of a lower concentration of reducing agent (such as DTT) compared to known sputum techniques. Previous techniques, typically using 0.1% (w/v) DTT, would enable cytokine analysis, but the quality of the cells would be too poor for simultaneous flow cytometric analysis. The gentler sample handling typically employed in the disclosed method, however, allows for the combined analysis of STAT phosphorylation and cytokine measurements in the same sputum sample. Use of 0.05% (w/v) DTT and gentle processing techniques have been found to also result in increased sensitivity of cytokine, chemokine and other biomarker measurements (See Example 2).

Exemplary biomarkers of inflammation include, but are not limited to, CC16, CXCL9, CXCL10, CXCL11, chemokine (C—C motif) ligand 2 (CCL2), CCL4, CCL5, GM-CSF, IFNγ, IL-1b, IL-2, IL-4, IL-5, IL-6, IL-8, IL-10, IL-12, IL-13, IL-17, interferon gamma-induced protein (IP)-10, MIP-1b, matrix metalloproteinase (MMP)-9, MMP-12, neutrophil elastase, TGFβ, tissue inhibitor of metalloproteinases (TIMP)-1, TNFα and thymic stromal lymphopoietin (TSLP), with a preference for CXCL9, CXCL10, CXCL11, CCL5 and IL-6. The biomarker may be a pro-inflammatory cytokine. Any biomarker of inflammation can potentially be measured in this way, the limiting factors being the volume of sample available, potential dilution effect making low levels of biomarkers of inflammation undetectable and the absence of the biomarker of inflammation in the original sample.

Different biomarkers of inflammation may be measured to assess the relationship with different combinations of pSTAT proteins. As exemplified herein and as an illustration, STAT1 phosphorylation was measured in relation to IL-1b, IL-6, IL-8, MIP-1b, CCL5, CXCL9, CXCL10 and CXCL11. The methods enable the exploration of patterns of inflammation in relation to phosphorylation of various STAT molecules. Different kinase inhibitors may have different effects on levels of biomarkers of inflammation (see below).

Biomarker (e.g. cytokine) levels can be measured in the sputum supernatant using, for example, Luminex® and enzyme-linked immunosorbent assay (ELISA) technology, in accordance with standard procedures in the art.

I. Assessing Kinase Inhibitors

Another embodiment provides a method for evaluating the efficacy and/or sensitivity of a kinase inhibitor. The method includes measuring STAT phosphorylation in a sputum sample using flow cytometry. The method allows the determination of drug effect on the human cell type of interest direct from the lung.

Evaluating the efficacy of a kinase inhibitor can mean determining whether the inhibitor is active in reducing or preventing phosphorylation of a STAT protein. This can be done using at least two different approaches; in vitro experiments in which sputum samples spiked with a known concentration of kinase inhibitor can be compared with those spiked with a comparator drug or placebo, this can be followed by in vivo testing of patients who have been dosed with the kinase inhibitor in clinical studies.

Known concentrations of a kinase inhibitor can be added prior to stimulating sputum cells (with a stimulator of STAT phosphorylation) in vitro to determine the concentration of inhibitor required to inhibit the stimulation by 50% (half maximal inhibitory concentration (IC50)). Multiple known concentrations of kinase inhibitor can be used to produce a dose response curve, i.e. to determine the in vitro dose required to reduce STAT phosphorylation by at least 30%, at least 50%, at least 70% or at least 85%. This in turn allows predictions regarding dose selection and administration to be made for future in vivo studies (see ‘Evaluating a suitable dose range and/or dosage regimen’ below). The in vivo studies would then be direct evidence of the IC50 in a clinical setting; the IC50 can be determined directly from patient samples after the relevant drug has been administered to the patient and that patient has subsequently produced a sputum sample for analysis. ‘Evaluating the efficacy’ of a kinase inhibitor can therefore mean determining the IC50 of the inhibitor with respect to the stimulation of STAT phosphorylation. A method of evaluating the efficacy of a kinase inhibitor is therefore an in vitro method, as the sputum samples have been previously removed from the subject and the entire evaluation process takes place outside the body on a processed sample. The method can, however, also be used in clinical studies, to obtain in vivo evidence of drug efficacy directly.

‘Evaluating the sensitivity’ of a kinase inhibitor refers to determining how effective an inhibitor is against STAT phosphorylation compared to another kinase inhibitor or placebo compound. The effect of an inhibitor may be significantly different from that of another kinase inhibitor or placebo compound; for example, one inhibitor may be substantially more potent in reducing or preventing phosphorylation of a STAT protein compared to another. An assay can be used to compare multiple compounds in order to assess their effects in comparison with one another, i.e. a new kinase inhibitor could be compared to a ‘gold standard’ or market leading compound. ‘Evaluating the sensitivity’ of a kinase inhibitor refers to determining the reduction in STAT phosphorylation that is achieved by the inhibitor, if any, compared to that achieved by an equivalent amount of another kinase inhibitor or placebo compound. It can encompass determining the IC50 of the inhibitor with respect to the stimulation of STAT phosphorylation and comparing it to that of another kinase inhibitor or placebo compound. A method of evaluating the sensitivity of a kinase inhibitor is therefore an in vitro method, as the sputum samples have been previously removed from the subject and the entire evaluation process takes place outside the body on a processed sample.

Still another embodiment provides a method for evaluating a suitable dose range and/or dosage regimen for a kinase inhibitor. The method includes measuring STAT phosphorylation in a sputum sample using flow cytometry. ‘Evaluating a suitable dose range and/or dosage regimen’ for a kinase inhibitor refers to determining the dose range and/or dosage regimen that would result in the inhibitor being active in reducing or preventing phosphorylation of a STAT protein. This could involve, for example, determining the IC50 of the inhibitor with respect to the stimulation of STAT phosphorylation, as described above. The purpose of the evaluation is typically to find the dose range and/or dosage regimen that would be suitable for use in vivo. In another embodiment, the in vitro data is used to determine a suitable dose range and/or dosage regimen for use in subsequent clinical studies. Thus, a method of the third embodiment may be an in vitro method. Typically, however, ‘evaluating a suitable dose range and/or dosage regimen’ for a kinase inhibitor involves carrying out a clinical study to determine both the effects in vivo of the kinase inhibitor directly and to determine the dose range and/or dosage regimen that would result in the inhibitor being active in reducing or preventing phosphorylation of a STAT protein (including the IC50). The in vitro dose response data can be combined with data regarding dose delivery methods, drug absorption rates and cellular uptake of the compound to determine a dose range and/or a dosage regimen for an in vivo study. A method of the third embodiment may therefore be an in vivo method, or it may involve both in vitro and in vivo steps, for example, it may involve a method of the second embodiment and/or a drug being administered to a person and at least part of the study being conducted inside a living organism, prior to a sputum sample being obtained and assessed. ‘Dosage regimen’ can mean the dose amount, the number of doses, the frequency or timing of administration and/or the period over which the inhibitor is to be administered.

Sputum samples may be taken from subjects who have been administered the kinase inhibitor by any route, but preferably by inhalation. The samples can then be assessed using a method of the third embodiment, i.e., include measuring STAT phosphorylation in a sputum sample using flow cytometry

Any kinase inhibitor can be used, including both selective and non-selective protein kinase inhibitors. As above, such inhibitors include, but are not limited to, PTK inhibitors, which include Src, Csk, Ack, Fak, Tec, Fes, Syk, Abl and Jak inhibitors, the latter including PF 956980, a known JAK3-selective inhibitor. MK2 inhibitors are also included. Preferably the kinase inhibitor is indicated or formulated for the treatment or prevention of a lung disease, particularly an inflammatory lung disease, and most particularly a lung disease(s) characterized by TH1 inflammation including, but not limited to, COPD, IPF and similar conditions. Thus, in an embodiment, a method of the second embodiment is for evaluating the efficacy and/or sensitivity of a kinase inhibitor in lung disease. Similarly, in an embodiment, a method of the third embodiment is for evaluating a suitable dose range and/or dosage regimen for a kinase inhibitor in lung disease. Preferably, the kinase inhibitor is a JAK inhibitor or a MK2 inhibitor.

The kinase inhibitor may be implicated or formulated for intravenous administration. In a preferred embodiment, the kinase inhibitor is implicated or formulated for inhalable or oral delivery. In a most preferred embodiment, the kinase inhibitor is implicated or formulated for inhalable delivery.

Inhaled delivery of kinase inhibitors may offer advantages for patients suffering from inflammatory lung diseases such as COPD, IPF and similar conditions, and the assays will assist in the clinical development of such compounds. Kinase inhibitors administered via the inhaled route are designed to be delivered direct to the lung and often have minimal or no systemic activity; hence, the whole blood assay that is known in the art for measuring STAT phosphorylation would not be relevant in these circumstances. Rather, the whole blood technique is relevant in the evaluation of oral drugs, which have a systemic drug distribution that results in measurable blood levels. The disclosed methods thus have use where the methods known in the art do not.

The methods of the second and third embodiments can be carried out by inducing STAT phosphorylation in the presence of the kinase inhibitor to be assessed. Thus, the procedure described above for the first embodiment (see section D) can be followed, but a kinase inhibitor can be added to a sputum cell sample (in vitro, second embodiment) or administered to a patient as part of a clinical study (in vivo, third embodiment).

For example, in the second embodiment, one pool of cells may be incubated with one or more stimulators of STAT phosphorylation (such as IFNγ or IL-6) alone, and a second pool of cells may be incubated with the one or more stimulators and the kinase inhibitor to be assessed. In the third embodiment one pool of cells may come from subjects who have been administered an inhaled kinase inhibitor and the other pool of cells may come from those who have received a different compound (e.g. placebo). Multiple pools of cells may be incubated with different stimulators of STAT phosphorylation and/or with different kinase inhibitors to be assessed. Any and all working combinations of STAT phosphorylation stimulators, STAT proteins to be measured and kinase inhibitors to be assessed are encompassed by the disclosed methods. In a preferred embodiment, measurement of STAT1 phosphorylation is made in sputum macrophage cells stimulated with IFNγ in the presence or absence of a kinase inhibitor, as illustrated herein by the JAK3-selective inhibitor, PF 956980. In other preferred embodiments, measurement of STAT1(Y701) or STAT3 phosphorylation is made in sputum macrophage cells stimulated with IFNγ in the presence or absence of a kinase inhibitor, as illustrated herein by a MK2 inhibitor.

For example, in the second embodiment 100 μl cells (200,000 cells) can be aliquoted into polystyrene tubes (such as flow cytometry tubes) or 90 μl cells+10 μl of the inhibitor to be assessed can be used (final concentration 10⁻⁵ M). As above, any suitable volume and number of cells can be aliquoted for analysis. Any suitable amount of the inhibitor can be added, the final concentration of inhibitor is typically in the range of 10⁻⁹ M to 10⁻³ M. In the third embodiment the dose of inhibitor administered to a subject could cover a similar range. In either embodiment, the exact range will depend upon the characteristics, potency and solubility of the compound being assessed. The skilled person would appreciate and know how to take account of such factors when deciding upon suitable concentrations to use.

As discussed above, a suitable amount of a stimulator of STAT phosphorylation (or DPBS as a negative control) is added to each sample, and the samples suitably incubated in a water bath, then centrifuged, the supernatant removed and the cell pellet resuspended in DPBS. Following further incubation in a water bath, the cells are ready for flow cytometric analysis, as described in sections F-G. Cytokine levels may also be measured in the sputum supernatant as per the first embodiment (section H).

All of the features described above for the first embodiment thus apply equally to other embodiments include the second and third embodiments.

The measurement of STAT phosphorylation as a biomarker in sputum has potential utility in drug development, and particularly the development of kinase inhibitors, notably those that are inhaled. In this regard, the disclosed methods can be used to assess the (inhaled) dose delivery of kinase inhibitors, and particularly JAK inhibitors and MK2 inhibitors. In particular, the pharmacokinetic and/or pharmacodynamic relationship can be explored.

The data (see Examples 1, 3 and 4) show spiked samples of kinase inhibitor were used to show inhibition of the stimulated cells (stimulation with IFNγ produced the STAT1 or STAT3 signal measured by flow cytometry). This could be regarded as the necessary pre-clinical in vitro step, whereby the method can be used to predict the dose or dose range of an oral or inhaled drug that may be required to inhibit the kinase mechanism in clinical studies. This assay, therefore, will be very useful to predict the design and conduct of future clinical studies, including dose setting, dose formulation and the likely clinical response.

In a clinical setting, the patients will have already inhaled the drug (or taken an oral or intravenous drug if applicable) and the sputum sample can be analyzed to show how effectively the drug is working in vivo.

After oral or intravenous administration, the known whole blood assay could be used, but if effects are sought solely in the lung, then only the sputum assay described herein would be relevant. Again, an inhaled drug, for lung diseases in particular, has advantages including local delivery to the site of action and usually a reduction in side effects commensurate with reduced systemic exposure.

The progression from laboratory (samples spiked with kinase inhibitor) to clinically-derived samples (after a patient has received a dose of a kinase inhibitor) is the potential utility of the biomarker method. It enables a seamless transition from pre-clinical tests of the compound on human cells to subsequent studies performed during clinical trials of the compound. This process is invaluable in clinical drug development and is known as “bench to bedside” drug development.

A fourth embodiment, therefore, provides the use of pSTAT as a biomarker for evaluating (i) the efficacy and/or sensitivity of a kinase inhibitor, and/or (ii) a suitable dose range and/or dosage regimen for a kinase inhibitor, the use including measuring STAT phosphorylation in a sputum sample using flow cytometry.

In a non-limiting example, a method for evaluating a suitable dose range and/or dosage regimen for a kinase inhibitor can include determining the IC50 of the inhibitor by measuring STAT phosphorylation in a series of test sputum samples including the kinase inhibitor, wherein each test sputum sample includes the kinase inhibitor at a different concentration. Methods of determining STAT phosphorylation are provided herein and include, for example, detecting or measuring STAT phosphorylation in a sputum sample by contacting the sputum sample or cells thereof with an antibody or antigen binding fragment thereof, or another ligand that binds specifically to a phosphorylated STAT and measuring the level of antibody-binding by flow cytometry.

Such a use may include any of the method steps set out above for the embodiments, in any combination.

A broad biomarker methodology can be used for measuring STAT phosphorylation in sputum. As sputum methods are highly specific and relatively uncommon, and the observations documented herein illustrate that a flow cytometry-based method can be used for sputum-derived measurements.

All of the features described herein (including any accompanying claims, abstract and drawings), and/or all of the steps of any method or process so disclosed, may be combined with any of the above embodiments in any combination, except combinations where at least some of such features and/or steps are mutually exclusive.

### EXAMPLES

### Example 1

### Measurement of STAT1 Phosphorylation and Pro-Inflammatory Cytokines in Induced Sputum Samples from COPD Subjects

Methods

Sputum Induction

Sputum samples were collected from 15 COPD subjects on three or four repeat visits (i.e., three to four samples per subject). In each case, the subject inhaled 3% (w/v) saline solution mist through the mouthpiece of an ultrasonic nebulizer for five minutes. Sputum mobilization techniques were utilized to assist with the production of a sputum sample, such as diaphragmatic breathing, huffs, percussion, vibrations and positive expiratory pressure techniques. The subject was asked to attempt to cough sputum into a sputum collection pot. If the FEV₁ fell by <10% after inhalation of 3% (w/v) saline, the participant was asked to inhale the next saline concentration (4% w/v) and repeat the procedure detailed above. Again if the FEV₁ fell by <10% after inhalation of 4% (w/v) saline, the participant was asked to inhale the next saline concentration (5% w/v) and repeat the procedure detailed above. The sputum collected after 15 minutes of nebulization (i.e., 3×5 minutes) was processed in the laboratory for flow cytometric analysis.

Sputum Processing

Induced sputum was kept on ice and processed as soon as possible but no more than two hours from collection. Sputum plugs were selected for processing and suitably transferred into a centrifuge tube. The volume of the selected sputum sample was noted and an equal volume of DPBS added. To liquefy the sample Sputolysin® was added to a final concentration of 0.05% (w/v). The tube was placed on a plate shaker (300 rpm) for 30 minutes at room temperature to disperse the cells. After 30 minutes the sample was mixed gently with a Pasteur pipette and left to shake for a further 15 minutes. The sample was centrifuged at 1200 rpm for 10 minutes at room temperature. Sputum supernatant was collected and used to measure cytokines/chemokines of interest.

Cell Counting

The cell pellet was resuspended in a known volume of DPBS. The cell suspension was diluted in 0.4% Trypan blue solution and loaded onto a hemocytometer in order to count the cells using microscopy. Total leucocyte count per milliliter of suspension was calculated by multiplying the total average leucocyte count by the dilution factor and multiplying by 10⁴.

Inducing STAT Phosphorylation

After the sputum sample had undergone liquefaction and a total cell count had been performed, the sputum cells were centrifuged at 1200 rpm for 10 minutes at room temperature and resuspended in DPBS at a concentration of 2×10⁶ cells/ml. The sample was left to rest undisturbed at 37° C. for one hour.

100 μl cells (200,000 cells) were aliquoted into polystyrene flow cytometry tubes or 90 μl cells+10 μl inhibitor for samples using the JAK inhibitor compound (JAK3-selective inhibitor, PF 956980; final concentration 10⁻⁵ M). 10 μl IFNγ (100 ng/ml) was added (final concentration 10 ng/ml). 10 μl DPBS was added to non-stimulated cells. The samples were incubated in a water bath at 37° C. for 20 minutes.

Sample Fixation and Permeabilization

The tubes were removed from the water bath and centrifuged at 258 g for five minutes at room temperature. The supernatant was removed and the cell pellet resuspended in 100 μl 4% (w/v) paraformaldehyde in DPBS. The samples were incubated in the water bath at 37° C. for 15 minutes.

The tubes were removed from the water bath and centrifuged at 258 g for five minutes at room temperature. The supernatant was removed and the cell pellet resuspended in 0.8 ml staining buffer (DPBS+2% human serum). The tubes were again centrifuged at 258 g for five minutes at room temperature. The supernatant was removed and the cell pellet resuspended in 0.35 ml 100% (v/v) ice-cold methanol (stored at −20° C.). The samples were then incubated on ice for 20 minutes.

Sputum Staining for Flow Cytometry

The tubes were centrifuged at 258 g for 5 minutes at room temperature. The supernatant was removed and the cell pellet resuspended in 0.8 ml staining buffer.

The tubes were centrifuged at 258 g for five minutes at room temperature. The supernatant was removed and the tubes blotted dry with laboratory tissue to ensure the removal of most of the liquid.

The cell pellets were resuspended in 100 μl staining buffer with the addition of either 20 μl staining buffer (unstained cells) or 20 μl (1.5 μg/ml) Alexa Fluor® 647 conjugated anti-pSTAT1 antibody (STAT stained cells) or isotype control (control cells) at the same concentration as pSTAT1. The samples were incubated at room temperature, covered in foil, for 30 minutes.

2 ml staining buffer was added and the tubes centrifuged at 258 g for five minutes at room temperature. The supernatant was removed and the cell pellet resuspended in 500 μl staining buffer ready for flow cytometric analysis.

Sputum Flow Cytometric Analysis

Gating strategy is shown in FIG. 2. Debris was gated out and the three distinct populations within P1 gated on with specific interest in P4 containing macrophages. The population (P2) to the immediate left of the macrophages (P4) represents neutrophils, and the small population (P3) at the bottom of the profile is unidentified.

Mean Fluorescence Intensity (MFI) Ratio of Stimulated/Non-Stimulated

Levels of STAT phosphorylation in the macrophage population were determined by taking the MFI of the stimulated sample and dividing by the MFI for the non-stimulated sample. A value of greater than one (>1) indicated positive staining.

Biomarker Analysis

The levels of pro-inflammatory cytokines and chemokines in sputum supernatants were analyzed using Luminex® and ELISA technology.

Results

As can be seen in FIG. 3, the level of intracellular pSTAT1 in macrophages was significantly increased in all samples after incubation with IFNγ (unstimulated MFI 120.7±23.92 vs stimulated MFI 196.7±33.97).

Incubation with IFNγ+JAK inhibitor resulted in complete inhibition of STAT1 phosphorylation (MFI 118.3±24.44).

FIG. 4, showing the mean values, illustrates the same trend.

There was no up-regulation of STAT1 in neutrophils.

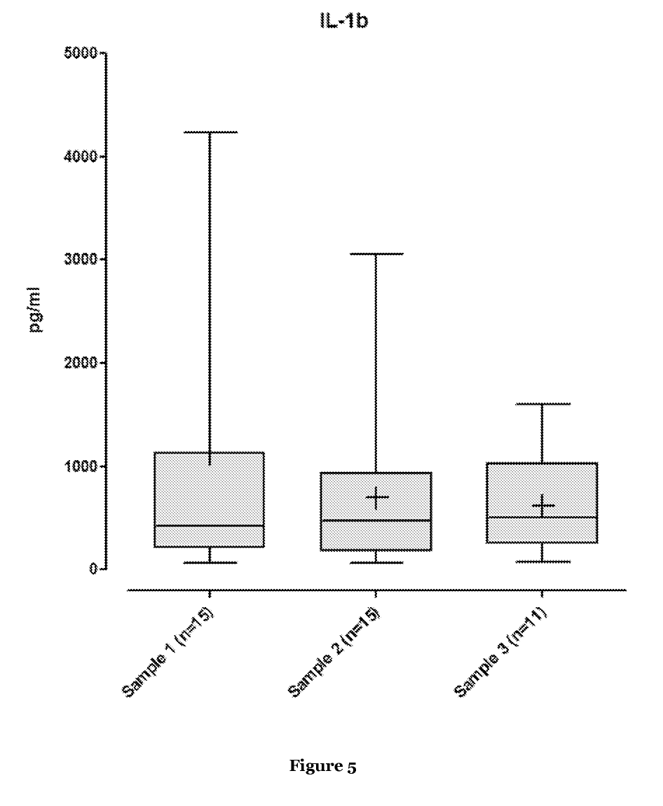

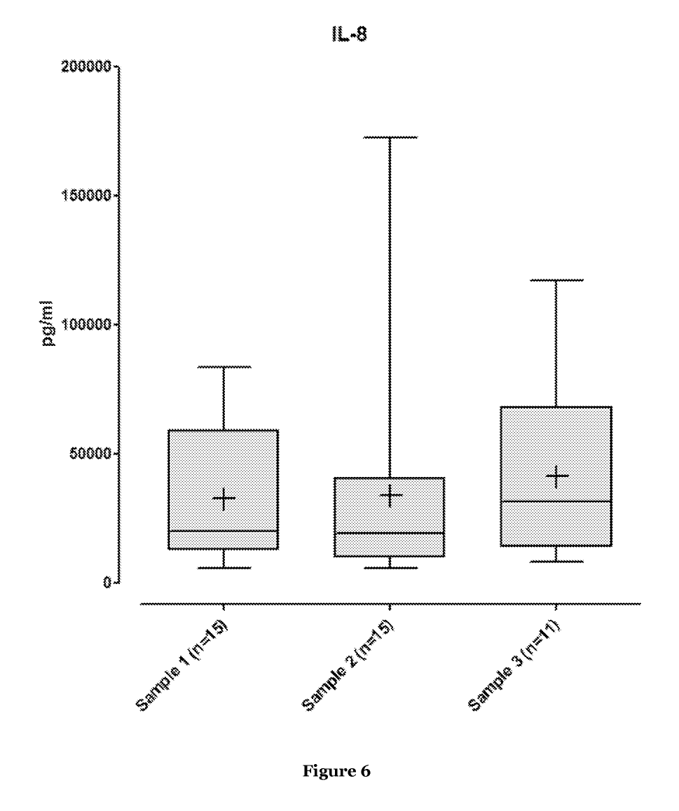

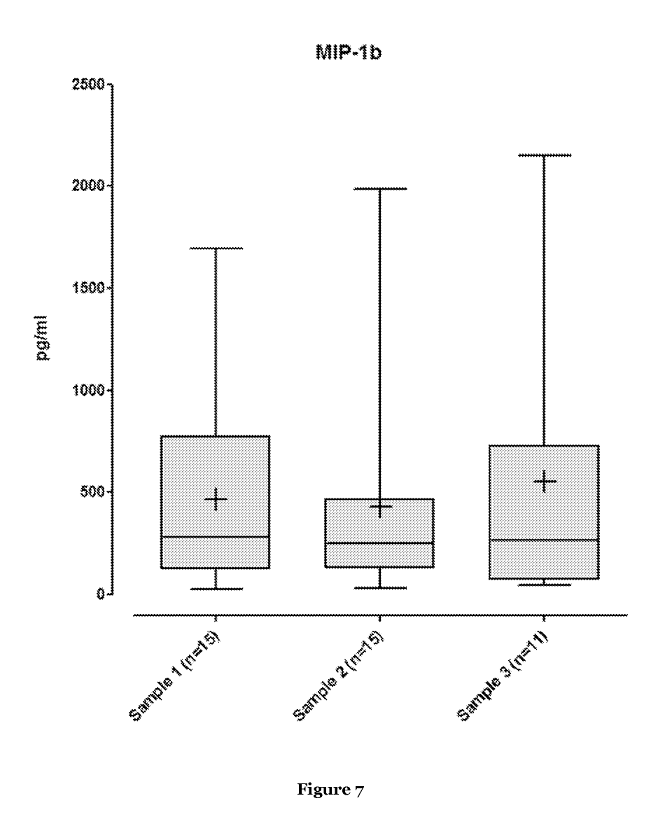

As can be seen in FIGS. 5-7, levels of pro-inflammatory cytokines (IL-1b, IL-8 and MIP-1b) were measurable in all sputum supernatants, with the levels being consistent over repeat visits.

There was a corresponding increase in inflammatory cytokines/chemokines, CXCL9, CXCL10, CXCL11, CCL5 and IL-6.

STAT1 phosphorylation and accompanying inflammatory cytokine levels can be reproducibly measured in sputum samples via these processing and analysis methods. The inhibition of STAT1 phosphorylation after IFNγ stimulation by a JAK inhibitor was also demonstrated as a measurable event. These data therefore confirm the validity and reproducibility of the assay system. These methods may be applicable for the identification and development of therapeutic compounds, particularly those delivered by inhalation direct to the lung.

The results of this study using a new flow cytometric technique for analyzing sputum samples indicate that macrophages play an important part in the JAK/STAT pathway of inflammation. Much previous work has focused on neutrophilic inflammation, but these data indicate that, not only are macrophages important, but they play a key role in the regulation of chronic airway inflammation.

The ability to use flow cytometry on sputum samples thus permits detailed analysis of the activation of signaling pathways in specific cell populations.

This method is useful for assessing the efficacy of treatments for COPD, for example, since sputum induction is less invasive than bronchoalveolar lavage, yet still provides information from the site of inflammation in COPD.

### Example 2

### Comparison of a Disclosed Sputum Processing Method with the Standard Method in the Art

Methods

Sputum Induction

In a separate study (to that described in Example 1), induced sputum samples were obtained as described in Example 1. In this study the subjects had an established clinical diagnosis of COPD (GOLD stage 1). Samples were obtained from 10 subjects.

Sputum Processing

Each sputum sample was divided into two halves, for differential processing. One half of each sample was processed according to established sputum techniques, as described in Pizzichini E et al., Eur Respir J. 1996 June; 9(6):1174-80 (involving the use of 0.1% (w/v) DTT). The other half of each sample was processed as described in Example 1 (incorporating 0.05% (w/v) DTT and a gentle handling technique).

Biomarker Analysis

Cytokine and chemokine levels in sputum supernatants were analyzed using Luminex® technology.

Cell Analysis

The divided sputum samples were then analyzed for cell viability, squamous contamination and differential cell counts, according to known procedures in the art.

Results

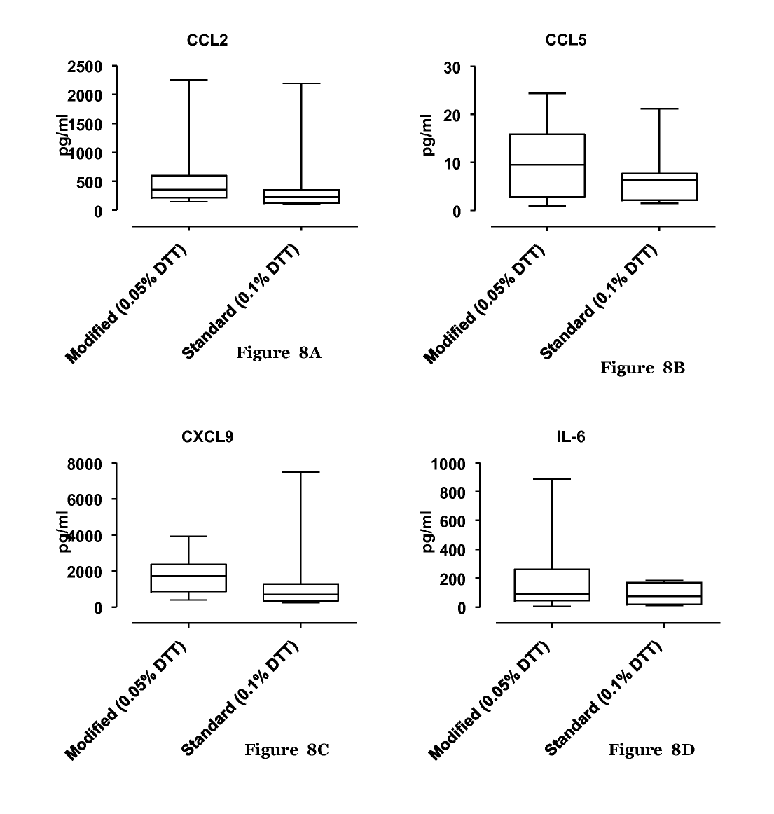

As shown in FIGS. 8A-D, cytokines (IL-6) (FIG. 8D) and chemokines (CCL2, CCL5 and CXCL9) (FIGS. 8A-C) were detected in all sputum supernatants. However, biomarker levels were increased in sputum supernatant processed according to the disclosed methods compared to those processed using the established techniques, with some biomarker levels being as much as three-fold greater.

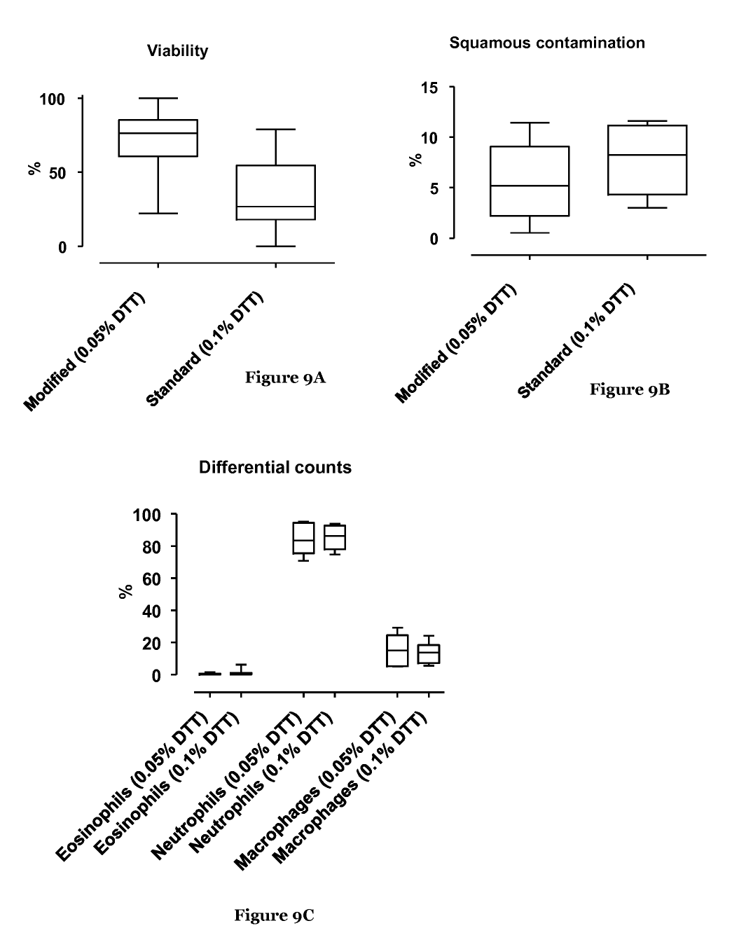

FIGS. 9A-C compares the cell data from the same induced sputum samples as shown in FIGS. 8A-D. It can be seen from FIGS. 9A-C that the disclosed sputum processing techniques significantly improved cell viability (FIG. 9A) compared to the established techniques; in this regard, the median % viability increased from 26% to over 75%. In these same samples the % squamous cell contamination (FIG. 9B) was reduced following processing with the disclosed techniques. Crucially the leucocyte differential count (FIG. 9C) was shown to be unaffected by the difference in processing techniques.

It has therefore been demonstrated that the disclosed method for induced sputum processing described herein (involving the use of 0.05% (w/v) DTT and gentle processing techniques) increases the sensitivity of biomarker measurements, increases cell viability and minimizes squamous cell contamination, whilst maintaining the integrity of cell differential counts.

### Example 3

### Measurement of STAT3 Phosphorylation in Induced Sputum Samples from COPD Subjects

Methods

The methods of Example 1 were repeated exactly, but this time measuring intracellular STAT3 phosphorylation using an Alexa Fluor® 647 conjugated anti-pSTAT3 antibody. In addition, a MK2 inhibitor was tested instead of a JAK inhibitor compound. Increasing concentrations of inhibitor were tested (1 μM, 10 μM and 100 μM); the same conditions otherwise applied.

Results

This study was designed to show that different pSTAT proteins can be measured using different antibody detection systems, and that different kinase inhibitors can be accessed via these respective pSTAT protein pathways. Where Example 1 demonstrated the use of an anti-pSTAT1 antibody to measure pSTAT1 induced by IFNγ (i.e. via the JAK-STAT pathway) in the presence and absence of a JAK inhibitor compound, this Example therefore demonstrates the use of an anti-pSTAT3 antibody to measure pSTAT3 induced by IFNγ (i.e. also via the JAK-STAT pathway), but this time in the presence and absence of a MK2 inhibitor, i.e. an inhibitor of the MAPK pathway.

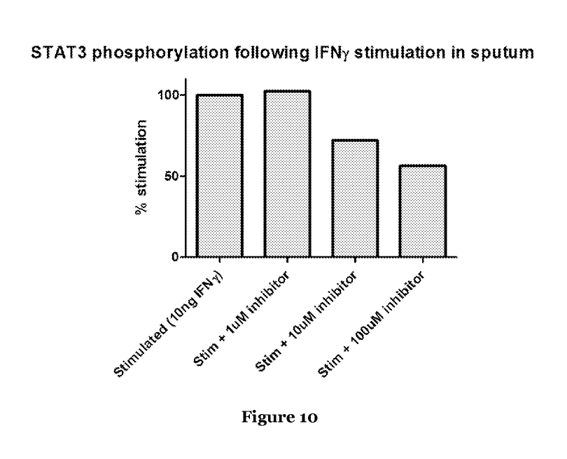

As can be seen in FIG. 10, the level of intracellular pSTAT3 in sputum macrophages was increased by 100% following incubation with IFNγ (% stimulation calculated as stimulated MFI/non-stimulated MFI×100).

Pre-incubation with increasing concentrations of MK2 inhibitor, followed by incubation with IFNγ, resulted in increasing inhibition of STAT3 phosphorylation.

The data show that STAT3 phosphorylation can be reproducibly measured in sputum samples via the disclosed processing and analysis methods. The data therefore confirm the validity and reproducibility of the assay system across different pSTAT proteins.

The inhibition of STAT3 phosphorylation was also demonstrated as a measurable event. Although phosphorylation was induced by IFNγ, i.e. via the JAK-STAT pathway, inhibition was achieved using a MK2 inhibitor, i.e. an inhibitor of the MAPK pathway.

The phenomenon of phosphorylation via the JAK-STAT pathway being inhibited via the MAPK pathway was investigated further in the study presented as Example 4. The data presented here nevertheless confirm the validity and reproducibility of the assay system across different pSTAT systems and different inhibitors of STAT phosphorylation. These data thus provide further evidence that the methods disclosed herein are applicable for the development of compounds, particularly those delivered by inhalation direct to the lung. The data also verify that, not only are macrophages important, but that they play a key role in the regulation of chronic airway inflammation.

### Example 4

### Performance of Sputum Processing in an Alternative (STAT1(Y701)) Pathway

Methods

Sputum Induction

Induced sputum samples were obtained as described in Example 1. In this study, however, the subjects were smokers. Sputum was collected from two subjects.

Sputum Processing

Sputum samples were processed as described in Example 1.

Cell Counting

Cells were counted as described in Example 1.

Inducing STAT1(Y701) Phosphorylation STAT phosphorylation was induced as described in Example 1. In this study, however, the inhibitor compound was a MK2 inhibitor at 10 ng/ml.

Sample Fixation and Permeabilisation

Samples were fixed and permeabilised as described in Example 1.

Sputum Staining for Flow Cytometry

Samples were stained as described in Example 1. However, in this study, and in order to specifically detect STAT1(Y701) phosphorylation, an Alexa Fluor® 647 conjugated anti-pSTAT1(Y701) antibody was used.

Sputum Flow Cytometric Analysis

Gating strategy was the same as that described in Example 1 (i.e. as shown in FIG. 2).

MFI Ratio of Stimulated/Non-Stimulated

Levels of STAT1(Y701) phosphorylation in the macrophage population were determined by taking the MFI of the stimulated sample and dividing by the MFI for the non-stimulated sample. A value of greater than one (>1) indicated positive staining.

Results

This study provides a further example of different pSTAT protein pathways being measured using different antibody detection systems, and of the inhibition of respective pSTAT systems by different kinase inhibitors. In this study, an anti-pSTAT1(Y701) antibody was used to measure STAT1 phosphorylation occurring specifically via the JAK-STAT pathway, in the presence and absence of a MK2 inhibitor (i.e. an inhibitor of the MAPK pathway). In this regard, STAT1 becomes tyrosine-phosphorylated at residue Y701 upon stimulation of the JAK/STAT pathway, and is therefore distinguishable from STAT1 phosphorylated at Serine 272, and Threonine 25, 222 and 334 upon stimulation of the MAPK pathway.

As can be seen in FIG. 11 (top graph), the level of intracellular pSTAT1(Y701) increased in macrophages when stimulated with IFNγ (unstimulated MFI 345.5 vs stimulated MFI 511) in sputum. Pre-incubation with a MK2 inhibitor reduced the STAT1(Y701) phosphorylation to MFI 399.5 in a dose-dependent manner. This trend was absent in neutrophils.

As can be seen in FIG. 11 (bottom graph), a reduction of 63% in phosphorylation of STAT1(Y701) was achieved with the highest dose of inhibitor (100 μM) in macrophages. As above, no such reduction was seen with neutrophils.

There results show that using IFNγ it was possible to achieve up-regulation of intracellular phosphorylation of STAT1(Y701). STAT1 becomes tyrosine-phosphorylated at Y701 upon stimulation of the JAK/STAT pathway and, as such, should not be measurable upon stimulation of the MAPK pathway where STAT1 becomes phosphorylated at Serine 272, and Threonine 25, 222 and 334. However when the MK2 inhibitor was added to sputum samples stimulated with IFNγ there was inhibition of pSTAT1(Y701).

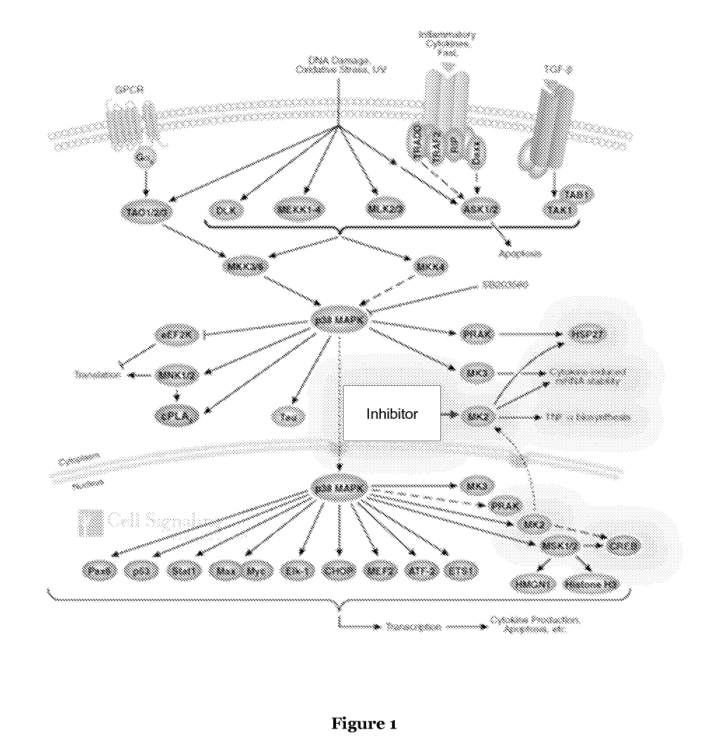

The issue when dealing with signaling pathways is that the level of cross-talk and interaction between various different pathways is largely an unknown factor. The p38MAPK pathway is known to be stimulated by a wide range of factors including lipopolysaccharide, osmotic shock and a range of cytokines that may also produce a similar effect. Similarly, other pathways, such as the JAK/STAT pathway or the NFκβ pathway, may interact or release factors which alter the activation of the p38MAPK pathway. It is entirely plausible that this was happening in the study presented here. As can be seen from FIG. 1, the number of downstream pathways leading off from p38 is large and, in order to see the effects of blocking one of these, focussed analysis endpoints may be required.

As the MK2 inhibitor compound is a peptide that is quickly taken up by cells, as with many inhaled drugs, it is thought highly likely that the inhaled dose will be taken up by respiratory epithelial cells within the lung. It is believed that the epithelial cells will therefore be the target cell population and that these cells will modify the inflammatory response. Depending upon the efficacy of the inhibitor compound this may lead to changes in the cellular composition of the induced sputum samples (i.e. total leucocyte count) and/or changes to levels of various secreted inflammatory markers (IL-8, growth regulated oncogene alpha (GRO-α)). Alternatively anti-inflammatory compounds may be taken up directly by macrophages (or other immune cells) and have a direct intracellular effect on these cell types.

I claim:

1. A method for measuring STAT phosphorylation comprising detecting STAT phosphorylation in a sputum sample by flow cytometry.

2. The method of claim 1, wherein the sample contains about 100,000-500,000 sputum cells.

3. The method of claim 1 further comprising a sputum processing step in which the sputum sample is treated with dithiothreitol (DTT) and optionally shaken at room temperature in an effective amount to disperse the cells without activating any inflammatory cells.

4. The method of claim 3, wherein the sputum processing step comprises adding DTT at a concentration of less than 0.1% (w/v) to the sample and gently shaking the mixture at room temperature for more than 15 minutes.

5. The method of claim 3 wherein the sputum processing step results in a cell viability of at least 70%.

6. The method of claim 3, wherein the sputum processing step further comprises inhibiting any proteases in the sample.

7. The method of claim 1, wherein the method comprises a STAT phosphorylation induction step in which the sample is treated with one or more cytokines.

8. The method of claim 1, wherein the STAT is STAT1, STAT2, STAT3, STAT4, STAT5A, STAT5B, or STATE, or a combination thereof.

9. The method of claim 1, wherein the STAT is STAT1 and phosphorylation is induced by IFNγ or IL-6, or a combination thereof.

10. The method of claim 1, wherein the method comprises inducing STAT phosphorylation in the presence of a kinase inhibitor.

11. The method of claim 10, wherein the kinase inhibitor is:
(i) indicated for administration by inhalation;
(ii) indicated for oral administration;
(iii) indicated for intravenous administration;
(iv) a selective or non-selective protein kinase inhibitor, a PTK inhibitor, a Jak inhibitor, or a MK2 inhibitor; and/or
(v) indicated for the treatment or prevention of a lung disease.

12. The method of claim 1, wherein the method comprises a cell permeabilisation step in which sputum cells are treated with 100% (v/v) methanol.

13. The method claim 1, wherein the flow cytometry is performed on:
(i) cells containing at least 4% macrophages; and/or
(ii) a sample volume of at least 100 μl.

14. The method of claim 1, wherein the method comprises inducing STAT1 phosphorylation in sputum macrophages using IFNγ, optionally in the presence of a kinase inhibitor.

15. The method of claim 1, further comprising measuring the level(s) of one or more biomarkers of inflammation in the sputum sample.

16. The method of claim 1, further comprising
adding an effective amount of DTT at a concentration of less than 0.1% (w/v) and optionally agitating or shaking the sample under conditions that release cells from mucus suitable for antibody staining while maintaining viability of at least 50% of the cells;
removing the supernatant;
fixing and permeabilizing the retained cells; and
staining the cells with an antibody that binds specifically to a phosphorylated STAT;
prior to detecting the level of antibody-binding by flow cytometry.

17. A method for evaluating the efficacy and/or sensitivity of a kinase inhibitor, the method comprising measuring STAT phosphorylation in a test sputum sample comprising the kinase inhibitor by flow cytometry.

18. The method of claim 17, wherein the level of STAT phosphorylation in the sample is compared to a control sputum sample wherein the STAT phosphorylation was measured in the absence of the kinase inhibitor, and wherein the kinase inhibitor is determined to modulate STAT phosphorylation when the level of STAT phosphorylation in the test sample is lower than in the control sample.

19. The method of claim 18, further comprising contacting the test sample with an effective amount of one or more cytokines in an effective amount to induce STAT phosphorylation in the cells of the sample.

20. A method for evaluating a suitable dose range and/or dosage regimen for a kinase inhibitor, the method comprising measuring STAT phosphorylation in one or more sputum samples by flow cytometry.
